(19) United States
(12) Patent Application Publication
(10) Pub. No.: US 2002/0068066 A1
(43) Pub. Date: Jun. 6, 2002

(54) Method for the treatment and prevention of dental caries

(76) Inventors: Wenyuan Shi, Los Angeles, CA (US); Sherie L. Morrison, Los Angeles, CA (US); Kham Trinh, Alhambra, CA (US); Letitia Wims, Culver City, CA (US); Li Chen, Los Angeles, CA (US); Maxwell H. Anderson, Seattle, WA (US)

Correspondence Address: David J. Meyer, 11355 W. Olympic Blvd., Los Angeles, CA, 90064, US

(21) Appl. No.: 09/881,823

(22) Filed: Jun. 15, 2001

Related U.S. Application Data
(63) Continuation-in-part of application No. 09/378,577, filed on Aug. 20, 1999.

(51) Int. Cl. A61K 39/09

(52) U.S. Cl. 424/185.1; 424/244.1

(57) ABSTRACT

Dental caries in man may be prevented or treated by oral ingestion of human or humanized murine monoclonal IgG and IgM antibodies that bind to surface antigens of cariogenic organisms, such as S. mutans. The genetically engineered monoclonal antibodies engage the effector apparatus of the human immune system when they bind to cariogenic organisms, resulting in their destruction. In a preferred embodiment, monoclonal antibodies to cariogenic organisms are produced by edible plants, including fruits and vegetables, transformed by DNA sequences that code on expression for the desired antibodies. The antibodies are applied by eating the plants.

Streptococcus mutans is believed to be the principal cause of tooth decay in man. When S. mutans occurs in large numbers in dental plaque, and metabolizes complex sugars, the resulting organic acids cause demineralization of the tooth surface. The result is carious lesions, commonly known as cavities. Other organisms, such as Lactobaccilli and Actinomyces are also believed to be involved in the progression and formation of carious lesions. Those organisms that cause tooth decay are referred to herein as “cariogenic organisms.”

Removal of the damaged portion of a tooth and restoration by filling can, at least temporarily, halt the damage caused by oral infection with cariogenic organisms. However, the “drill and fill” approach does not eliminate the causative bacterial agent. Proper oral hygiene can control the accumulation of dental plaque, where cariogenic organisms grow and attack the tooth surfaces. However, dental self-care has its limits, particularly in populations that are unable to care for themselves, or where there is a lack of knowledge of proper methods of self care. Administration of fluoride ion has been shown to decrease, but not eliminate the incidence of dental caries.

In view of the overwhelming evidence of the involvement of cariogenic organisms in the pathogenesis of dental caries, it is not surprising that there have been a number of different attempts to ameliorate the condition using traditional methods of anti-microbial therapy. The disadvantage of antimicrobial agents is that they are not selective for cariogenic organisms. Administration of non-specific bacteriocidal agents disturbs the balance of organisms that normally inhabit the oral cavity, with consequences that cannot be predicted, but may include creation of an environment that provides opportunities for pathogenic organisms. In addition, long term use of antimicrobial agents is known to select for organisms that are resistant to them. Hence long term and population-wide use of antimicrobial agents to prevent tooth decay is not practical.

Vaccination of humans to elicit an active immune response to S. mutans, or other cariogenic organisms, is also not a practical solution at this time. One drawback of this approach is that vaccination elicits production of predominantly IgG and IgM antibodies, but they are not secreted into saliva. The majority of antibodies present in saliva are of the IgA isotype, which can bind to, but cannot activate lymphocytes or complement components to kill bacteria. Accordingly, vaccination is not believed likely to be capable of producing antibodies that can trigger the immune system to kill cariogenic organisms in the mouth. There is no known method for selectively increasing the titer of vaccination induced antibodies of the IgG or IgM isotypes in the oral cavity.

There have been a number of reported attempts to passively immunize patients to S. mutans using monoclonal IgA antibodies raised in mice to prevent tooth decay in animals and in man. Because IgA is a multivalent antibody, a single molecule of IgA can bind to several different antigenic sites, resulting in clumping of bacteria. However, binding of IgA to bacterial surface antigens does not kill the bacteria. Rather, clumping is believed to hinder the ability of bacteria to bind to tooth surfaces. Another drawback of this approach is that repeated administration of murine (i.e., heterologous) antibodies to humans has the potential to evoke an immune response to the antibodies.

Unlike IgA antibodies, antibodies of the IgG and IgM classes have bacteriocidal effects. Binding of IgM or IgG antibodies to antigens present on the surface of cariogenic organisms may result in the destruction of the bacterial cells by either of two presently known separate mechanisms: complement mediated cell lysis and antibody-dependent cell-mediated cytotoxicity. In either case, antibodies that selectively bind to certain microbial organisms target just those cells for destruction by the immune system. Both complement mediated cell lysis and antibody-dependent cell mediated cytotoxicity are part of the humoral immune response that is mediated by antibodies of the IgG and IgM classes.

In order to elicit the desired cytotoxic effect of antibody binding, monoclonal antibodies to cariogenic organisms must be recognized by the human immune system. There are a number of different technologies by which antibodies that will trigger a response from the human immune system can be produced. One example is producing a chimeric antibody using a nucleic acid construct that codes for expression of a human antibody modified to incorporate sequences encoding the variable domain from a different source. Another method utilizes a phage display to determine the actual binding sites of the monoclonal antibody, the complimentarily determining regions (CDRs), and then grafting the CDRs onto the framework regions of the variable domains of a human immunoglobulin by site directed mutagenesis. Still other methods, known in the art, which allow the production of antibodies capable of engaging the humoral immune systems include: 1) Immunizing mice which have been genetically altered to produce human antibodies; 2) Immunizing isolated human B cells in vitro and then going through a cell fusion procedure to produce a hybridoma that secretes the antibody; and 3) Isolating B cells from humans with acute infection and producing an antibody generating hybridoma.

Production and administration of such genetically engineered humanized or human monoclonal antibodies to treat dental caries in man poses issues requiring innovative solutions. Prior art methods for production of monoclonal antibodies involve growing hybridomas in culture media, followed by extraction and purification of the desired antibody. These steps are significantly simplified in a preferred embodiment of the invention by expressing the antibodies in edible plants or animals. The antibodies are administered upon oral ingestion of plant or animal products, such as fruits, vegetables or milk wherein the antibodies are not denatured. This mode of administration has the potential for obviating compliance issues in ameliorating tooth decay.

U.S. patent application Ser. No. 09/378,247 discloses three murine monoclonal antibodies specific to S. mutans: SWLA1, SWLA2 and SWLA3. Development of an effective immunological method for the treatment and prevention of dental caries requires preparation of monoclonal antibodies genetically engineered to both express monoclonal antibodies specific to S. mutans and engage the effector apparatus of the human immune system.

### SUMMARY OF THE PREFERRED EMBODIMENTS

Dental caries may be prevented or treated by oral ingestion of human or humanized murine monoclonal IgG and IgM antibodies that bind to surface antigens of cariogenic organisms, such as S. mutans. The genetically engineered monoclonal antibodies engage the effector apparatus of the human immune system when they bind to cariogenic organisms, resulting in their destruction. In a preferred embodiment, monoclonal antibodies to cariogenic organisms are produced by edible plants, including fruits and vegetables, transformed by DNA sequences that code for expression of the desired antibodies. The genetically engineered monoclonal antibodies are applied by eating the transformed plants.

We have now isolated and sequenced the nucleotide sequences encoding the variable regions of monoclonal antibodies specific to S. mutans. When expressed, monoclonal antibodies encoded by these sequences bind specifically to S. mutans. Through the use of recombinant techniques, the variable regions of the monoclonal antibodies have been linked to the constant region of human antibodies thereby generating a chimeric monoclonal antibody that specifically binds S. mutans. This chimeric monclonal antibody is directed specifically to surface antigens of cariogenic organisms which generates an effector response from the immune system upon binding to the target organism.

### DETAILED DESCRIPTION OF THE PREFERRED EMBODIMENTS

1. Preparation of Monoclonal Antibodies

The monoclonal antibody technique permits preparation of antibodies with extraordinary specificity. Monoclonal antibodies that bind to specific molecular structures can be produced using what are today considered standard techniques.

The monoclonal antibodies that may be used in this invention are those that are directed to surface antigens of cariogenic organisms. Surface antigens are substances that are displayed on the surface of cells. Such antigens are accessible to antibodies present in body fluids. In the context of the present invention, surface antigens of cariogenic organisms are present on the surface of organisms that cause dental caries. While the role of bacterial activity in the genesis of carious lesions is well defined, establishing a cause and effect relationship between a particular organism and the occurrence of dental caries has not been completely successful. To date, only S. mutans has been definitively associated with dental caries. However, species of the Lactobaccili and Actinomyces are also believed to be involved, particularly with the active progression of carious lesions. Any organism that can produce a carious lesion is a potential target for the monoclonal antibodies prepared and used in accordance with this invention.

A further requirement of the monoclonal antibodies that may be used in the practice of the present invention is that they are selective for cariogenic organisms. Monoclonal antibodies directed to antigens present on cariogenic as well as non-cariogenic organisms may produce non-specific alterations in the makeup of the flora within the oral cavity. The consequences of such changes are not understood.

Accordingly, the preferred monoclonal antibodies selectively bind to surface antigens of cariogenic organisms. That is to say, the preferred monoclonal antibodies bind specifically to organisms that cause dental caries.

It should be clearly understood that the scope of the present invention is not limited to the prevention of tooth decay in man. Monoclonal antibodies in accordance with the present invention can be genetically engineered to engage the effector response of the immune system of other mammals, such as those that are domesticated as pets.

Monoclonal antibodies can be prepared by immunizing mice or other mammalian hosts with cell wall material isolated from cariogenic organisms. In a preferred embodiment, the cariogenic organisms are type c S. mutans (ATCC25175). The immunogenecity of molecules present in cell walls may be enhanced by a variety of techniques known in the art. In a preferred embodiment, immunogenecity of such molecules is enhanced by denaturation of the isolated cell material with formalin. Other techniques for modifying cell wall proteins to enhance immunogenecity are within the scope of this invention. Typically, hosts receive one or more subsequent injections of isolated bacterial cell fragments to increase the titer of antibodies prior to sacrifice and cloning.

Spleen cells from hosts are harvested. The NSI/Ag4.1 mouse myeloma cell line was used as the fusion partner and grown in spinner cultures in 5% CO₂ at 37° C. and maintained in log phase of growth prior to fusion. Hybridomas were produced according to the procedure reported by Kohler et al. Nature, 256:495-497, (1975). Hybrids were selected in media containing HAT (100 μg Hypoxanthine, 0.4 μM Aminopterin; 16 μM Thymidine). HT (100 μg Hypoxanthine; 16 μM Thymidine) was maintained in the culture medium for 2 weeks after aminopterin was withdrawn. OPI (1 mM oxaloacetate, 0.45 mM pyruvate and 0.2 U/ml bovine insulin) was added as additional growth factors to the tissue culture during cloning of the hybridomas. The hybridomas were further cloned by limiting dilution using techniques that have become standard since the pioneering work of Kohler and Milstein. In a preferred embodiment, surviving hybridomas were screened for antibody directed to cariogenic organisms by ELISA assay against microtiter plates coated with formalinized bacterial cell material. Positive supernatants were subjected to further screening to identify clones that secrete antibodies with the greatest affinity for the cariogenic organisms. In a preferred embodiment, clones with titers at least three times higher than background are screened again using immunoprecipitation with denatured cell wall material from S. mutans. In a preferred embodiment, three clones were identified which bound detectably only to S. mutans strains ATCC25175, LM7, OMZ175 and ATCC31377. These clones were deposited with the American Type Culture Collection, receiving Deposit Numbers HB 12599 (SWLA1), HB 12560 (SWLA2), and HB 12558 (SWLA3). U.S. patent application Ser. No. 09/378,247.

There are various ways to obtain nucleic acid sequences that code for expression of human or humanized monoclonal antibodies specific for the surface antigens of cariogenic organisms: 1) Isolating murine hybridomas which produce monoclonal antibodies against cariogenic organisms and cloning murine genes that code for expression of those antibodies; 2) Using purified cariogenic organisms to screen a phage display random library made from human B lymphocytes to obtain genes that encode antibodies specific for cariogenic organisms; 3) Isolating human hybridomas that produce monoclonal antibodies against cariogenic organisms, using B lymphocytes recovered from heavily infected patients and cloning the human genes encoding these antibodies; or 4) Immunizing human B lymphocytes and spleen cells in vitro using purified cariogenic organisms, followed by fusion to form hybridomas to create immortal cell lines. The techniques required are known to those skilled in the art and are not limited to the methods described herein.

2. Preparation of Monoclonal Antibodies Capable of Eliciting an Effector Response from Human Immune System

Previous efforts to develop an immunological method for the prevention of dental caries employed heterologous antibodies. For example, Lehner, U.S. Pat. No. 5,352,446, refers to use of monoclonal antibodies to S. mutans surface antigens raised in mice in inhibiting the proliferation of those bacteria in monkeys. More recently, Ma et al. Nature Medicine, 45(5) 601-6 (1998), reported similar results in humans, using a genetically engineered secretory monoclonal murine antibody to S. mutans expressed in tobacco plants. Drawbacks to this approach include 1) administration may aggregate the offending organisms, but not kill them because the non-human antibodies do not effectively engage the human immune response; and 2) repeated administration of the antibody may elicit an immune response from the patient to the antibody. A preferable approach is to use recombinant techniques to prepare chimeric antibody molecules directed specifically to surface antigens of cariogenic organisms, that will also elicit an effector response from the immune system of the mammal treated therewith upon binding to the target organism. This can be accomplished by inserting variable regions from murine monoclonal antibodies that are specific to cariogenic organisms into antibodies of the IgG and/or IgM classes from the mammal to be treated. It is also possible to generate antibodies that utilize just the complementarity determining regions (CDRs) of a murine monoclonal antibody specific to cariogenic organisms. Through known recombinant techniques, the CDRs are transferred into the immunoglobulin's variable domain.

Methods are also known for generating the antibody directly, for example: 1) Immunizing mice which have been genetically altered to produce human antibodies; 2) Immunizing isolated human B lymphocytes in vitro and then going through a cell fusion procedure to produce a hybridoma that secrets the antibody; and 3) Isolating B Imphocytes from humans with acute infection and producing an antibody generating hybridoma. The techniques required are known to those skilled in the art. Because each method produces a human antibody, the antibodies are capable of engaging the humoral immune effector systems upon binding to their specific antigens.

In the presently preferred embodiment of the invention, chimeric antibodies specific to S. mutans were generated. Using PCR or Southern blot techniques, DNA fragments encoding the variable domains of murine hybridomas secreting antibody specific to cell surface antigens of cariogenic organisms were isolated. Using gene cloning techniques, the variable regions were joined to the constant regions of human immunoglobulins. The result of this genetic engineering is a chimeric antibody molecule with variable domains that selectively bind to surface antigens of cariogenic organisms, but which interacts with the human immune effector systems through its constant regions.

3. Administration of Monoclonal Antibodies

In order to prepare a sufficient quantity of monoclonal antibodies for clinical use, the desired cell line, transfected with sequences encoding the immunoglobulin, must be propagated. Existing technology permits large scale propagation of monoclonal antibodies in tissue culture. The transfected cell lines secrete monoclonal antibodies into the tissue culture medium. The secreted monoclonal antibodies were recovered and purified by gel filtration and related techniques of protein chemistry.

In experimental studies, monoclonal antibodies to S. mutans have been applied directly to the surface of teeth. Application by ingestion of mouthwash, or by chewing gum has also been proposed. A presently preferred alternative is to express the chimeric monoclonal antibodies of the present invention in edible plants, such as banana or broccoli. Eating plants transformed in accordance with this invention will result in application of the antibodies to cariogenic organisms present on tooth surfaces, and elsewhere in the mouth. It is also contemplated that other organisms, both plant and animal, may be transformed to express the monoclonal antibodies described herein, so that such antibodies may be ingested, for example, by drinking milk.

### BRIEF DESCRIPTION OF THE DRAWINGS

The present invention is now described, by the way of illustration only, in the following examples which refer to the accompanying FIGS. 1-8, in which:

FIG. 1 shows the DNA sequences (SEQ ID NOS: 1 and 3) encoding the variable regions of the chimeric antibody (TEDW) specific to S. mutans derived from SWLA1 cells together with the predicted amino acid sequences (SEQ ID NOS: 2 and 4).

FIG. 2 shows the DNA sequences (SEQ ID NOS: 5 and 7) encoding the variable regions of the chimeric antibody (TEFE) specific to S. mutans derived from SWLA2 cells together with the predicted amino acid sequences (SEQ ID NOS: 6 and 8).

FIG. 3 shows the DNA sequences (SEQ ID NOS: 9 and 11) encoding the variable regions of the chimeric antibody (TEFC) specific to S. mutans derived from SWLA3 cells together with the predicted amino acid sequences (SEQ ID NOS: 10 and 12).

FIG. 4 shows the DNA sequence (SEQ ID NO: 13) encoding an aberrant light chain variable region derived from SWLA1 cells together with the predicted amino acid sequence (SEQ ID NO: 14).

FIG. 5 shows the DNA sequence (SEQ ID NO: 15) encoding a non-effective heavy chain variable region derived from SWLA1 cells together with the predicted amino acid sequence; (SEQ ID NO: 16).

FIG. 6 shows the DNA sequence (SEQ ID NO: 17) encoding an aberrant heavy chain variable region derived from SWLA1 cells together with the predicted amino acid sequence; (SEQ ID NO: 18).

FIG. 7 shows the DNA sequence (SEQ ID NO: 19) encoding an aberrant heavy chain variable region derived from SWLA2 cells together with the predicted amino acid sequence; (SEQ ID NO: 20).

FIG. 8 shows light and florescent microscope images of chimeric antibody TEDW binding to S. mutans.

### EXAMPLES

1. Producing Murine Monoclonal Antibodies Against S. mutans

Type c S. mutans strain ATCC25175 were grown to log phase in BHI medium and washed twice with phosphate buffered saline, pH 7.2 (PBS), by centrifugation at 3000× g for 5 min. The pellet was resuspended in 1% formalin/0.9% NaCl, mixed at room temperature for 30 min and washed twice with 0.9% NaCl. BALB/c mice (8-10 weeks) were immunized intraperitoneaIly with 100 μl of the antigen containing approximately 10⁸ whole cells of formalinized intact S. mutans bacteria emulsified with Freund's incomplete adjuvant (FIA). After 3-5 weeks, mice received a second dose of antigen (10⁸ whole cells of bacteria in FIA). Three days prior to sacrifice, the mice were boosted intravenously with 10⁸ whole cells of bacteria in saline.

Spleen cells from hosts were harvested. The tissue culture medium used was RPMI 1640 (Gibco) medium supplemented with 2 mM L-glutamine, 1 mM sodium pyruvate, and 10 mM HEPES and containing 100 μg/ml penicillin and 100 μg/ml streptomycin with 10% fetal calf serum. The NSI/Ag4.1 mouse myeloma cell line was used as the fusion partner and grown in spinner cultures in 5% CO₂ at 37° C. and maintained in log phase of growth prior to fusion. Hybridomas were produced according to the procedure reported by Kohler et al. Nature, 256:495-497, (1975).

Hybrids were selected in medium containing HAT (100 μg Hypoxanthine, 0.4 μM Aminopterin; 16 μM Thymidine). HT (100 μg Hypoxanthine; 16 μM Thymidine) was maintained in the culture medium for 2 weeks after aminopterin was withdrawn. OPI (1 mM oxaloacetate, 0.45 mM pyruvate and 0.2 U/ml bovine insulin) was added as additional growth factors to the tissue culture during cloning of the hybridomas. The hybridomas were further cloned by limiting dilution using techniques that have become standard since the pioneering work of Kohler and Milstein.

The following approach was used for screening for species-specific monoclonal antibodies against S. mutans. The initial screening was performed using an ELISA assay, which selects for the culture supernatants containing antibodies that bind to S. mutans. Formalinized bacteria were diluted in PBS to OD₆₀₀=0.5, and added to duplicate wells (100 μl) in 96 well PVC ELISA plates preincubated for 4 h with 100 μl of 0.02 mg/ml Poly-L-lysine. These antigen-coated plates were incubated overnight at 4° C. in a moist box then washed 3 times with PBS and blocked with 0.5% fetal calf serum in PBS and stored at 4° C. 100 μl of mature hybridoma supernatants were added to the appropriate wells of the antigen plates, incubated for 1 h at room temperature, washed 3 times with PBS-0.05% Tween 20, and bound antibody was detected by the addition of polyvalent goat-anti-mouse IgG antibody conjugated with alkaline phosphatase diluted 1:1000 with PBS-1% fetal calf serum. After the addition of the substrate, 1 mg/ml p-nitrophenyl phosphate in carbonate buffer (15 mM Na₂CO₃, 35 mM NaH₂CO₃, 10 mM MgCl₂ pH 9.6), the color development after 15 min was measured in an EIA reader at 405 nm. The positive supernatants (3 fold higher than control) were then subjected to the immunoprecipitation assay (mixing 100 μl bacteria with 100 μl supernatant) to screen for those with strong positive reactivity with S. mutans. The deposited clones (ATCC HB 12599, HB 12560, and HB 12558) were prepared according to this method.

2. Preparation of Hybridoma Lines for Cloning of V Regions.

A. Isotyping

The hybridoma supernatants were isotyped with a Pharmigen Isotyping Kit (BD Pharmigen, San Deigo, Calif.). 200 μl of isotype specific rat anti-mouse antibody was diluted in 800 μl of coating buffer and 50 μl of each reagent was added to 10 wells of a 96 well polystyrene ELISA plate. Plates were incubated overnight at 4° C. The plate was washed four times with washing buffer, 0.05% Tween-20 in PBS, and the remaining contents shaken out and the plate blotted dry on a paper towel. 200 μl of blocking solution, 1% BSA in PBS, was added to each well and the plate was incubated at room temperature for 30 minutes. Plates were again washed four times and the contents shaken out. 50 μl of hybridoma supernatant was added to the appropriate wells. Positive controls from the kit were added to the appropriate wells. The plate was incubated at room temperature for one hour. The plate was washed five times with washing buffer and the plate was blotted dry. One phosphatase substrate tablet was dissolved in 5.0 ml of p-NNP substrate diluent. 50 μl of substrate solution was added to each well and the plate was incubated for 40 minutes. The plate was read at 405 nm on a Dynatech MR 700 microplate reader. SWLA1, SWLA2 and SWLA3 were all determined to be γ2a,κ (IgG).

B. Biosynthetic Labeling

Cells were washed twice in methionine-free, Dulbecco's modified Eagle's medium (DME, Irvine Scientific) supplemented with non-essential amino acids (Grand Island Biological) and glutamine (29.2 μg/ml). Cells were labeled in 1 ml of DME with 15 μCi [³⁵S]methionine (Amersham, Arlington Heights, Ill.). All labels were done using 3×10⁶ cells.

For labeling of secretions, ³⁵S-methionine was added to 15 μC/ml and cells were labeled for 3 hours at 37° C. Cells were harvested on to ice and pelleted by centrifugation. To isolate secreted IgG, the radioactive medium was transferred to a clean tube.

For measurement of cytoplasmic IgG, the cell pellet was lysed in 0.5 ml of NDET (1% NP40, 0.4% deoxycholate, 66 mM EDTA and 10 mM Tris, pH 7.4), nuclei were pelleted by centrifugation, and the cytoplasmic lysate transferred to a fresh tube. To immunoprecipitate the secreted or cytoplasmic IgG, rat anti-mouse kappa sepharose (prepared in the laboratory) was added. The samples were mixed overnight at 4° C., washed in NDET and then washed with dH₂O. The precipitates were resuspended in sample buffer (25 mM Tris, pH 6.7, 2% SDS, 10% glycerol, 0.008% bromophenol blue), and the antibodies were eluted from the sepharose by boiling. The samples were analyzed by SDS-PAGE and autoradiography without reduction on 5% phosphate gels; samples treated with 2-mercaptoethanol were analyzed using 12% tris-glycine gels. Results indicate that all three clones made the same size heavy chain but different size light chains. All three hybridomas were subcloned to ensure homogenous cell populations.

C. Subcloning

The hybridomas were subcloned on soft agar. A 60 mm petri dish was coated with 5 ml of growth media plus 10% J774.2 (a murine macrophage cell line) supernatant plus 0.24% agarose (Sigma). The agarose was allowed to harden and a single cell suspension of hybridoma cells mixed with agarose was layered on top. When colonies were about 64 cells in size, they were overlaid with rabbit anti-mouse γ2a specific antiserum mixed with agarose. An immune precipitate forms over and partially obscures those clones secreting γ2a,κ antibody. Colonies making the most antibody, were identified and moved up to bulk culture where they were once again biosynthetically labeled.

3. Cloning Variable Regions from SWLA Cells

The basic protocol for cloning the variable regions from SWLA cells is outlined below followed by its application to specific SWLA hybridomas.

(i) Murine mRNA is made from about 5×10⁶ of both the original and subcloned cells using the Microfast Track Kit from Invitrogen.

(ii) First strand cDNA is made using oligonucleotides that prime the 5′ of the light or heavy chain constant region or that prime to the polyA tail of mRNA.

Basic Protocol:

(a) Half of the cDNA is resuspended in 20 μl RNase free dH₂O

(b) 2 μl of 0.5 mg/ml primer is added; the mixture is incubated@60° C. for 10 minutes

(c) The sample is cooled on ice; 8 μl of 5× first strand cDNA buffer, 2 μl of RNasin (Promega), 4 μl of 5 mM dNTP, and 0.5 μl of AMV Reverse Transcriptase are then added

(d) The sample is incubated@42° C. for 1 hour

(iii) PCR amplification is done with a number of different light or heavy chain signal peptide primers and primers that hybridize 5′ of the light or heavy chain constant region.

PCR Conditions:

(a) Denature@94° C. for 40 sec.

(b) Anneal@60° C. for 40 sec.

(c) Extend@72° C. for 40 sec.

(d) Amplify for 30 cycles

(e) Final Extension at 72° C. for 10 min.

(iv) The resulting PCR products are cloned into Invitrogen's PCR2.1 vector via the TOPO Cloning Kit.

(v) Individual clones were sent out for sequencing. The results were analyzed for an open reading frame (ORF) and compared with the known database to ensure that the sequence cloned is a variable region.

(vi) To check for PCR induced nucleotide alterations in the sequence, steps III to V were repeated so that the sequence of different clones from independent PCR reactions can be compared to ensure the accuracy of the sequence. The sequence data are also used to determine the sequence of the J region primer that needs to be used.

(vii) The variable region was cloned into the proper light (human kappa) or heavy chain (human IgG1) expression vector.

Typical Variable Region Ligation Protocol:

(a) 1 μg each of vector and insert was cut with either NheI/EcoRV or SalI/EcoRV

(b) The relevant fragments were then isolated using Qiagen's Gel Extraction Kit

(c) The ligation reaction used 4 μl out of 30 μl of vector sample and 6 μl out of 30 μl of insert sample

(d) 4 to 5 units of T4 DNA ligase was then added for a 20 μl final reaction volume

Typical Transformation Protocol:

(a) An aliquot of HB101 chemically competent E. coli cells were thawed on ice

(b) The entire ligation reaction was then added to the cells and incubated on ice for 15 minutes

(c) The cells were then heat shocked@42° C. for 1 minute

(d) 1 ml of LB was added to the cells and the tube was shaken@37° C. for 1 hour

(e) The tube was spun down@8000 RPM for 2 minutes

(f) All but 100 μl of supernatant was removed

(g) The cells were resuspended, plated onto LB+AMP plates, and incubated@37° C. overnight

B. Cloning the Variable Regions from SWLA1 Cells

In attempting to clone the light chain variable region (VL), PCR product was found using signal peptide primer 442 with constant region primer 450 as shown below. Previous studies have determined that 442 also primes to an endogenous aberrant or non-productive VL, SWLA1 Aberrant VL (SEQ ID NO: 13). Knowing this, attempts were made to enrich for non-aberrant transcripts by restriction digesting the PCR product with PflMI, which recognizes a specific sequence in the aberrant VL. Eventually, one variable region sequence was found to have an ORF. The final PCR product SWLA1 VL (SEQ ID NO: 1) was generated with primer 442 and J region primer 453 as shown below and inserted into the appropriate expression vector. The resulting human kappa expression vector carrying the VL from SWLA1 is named 5936 pAG.

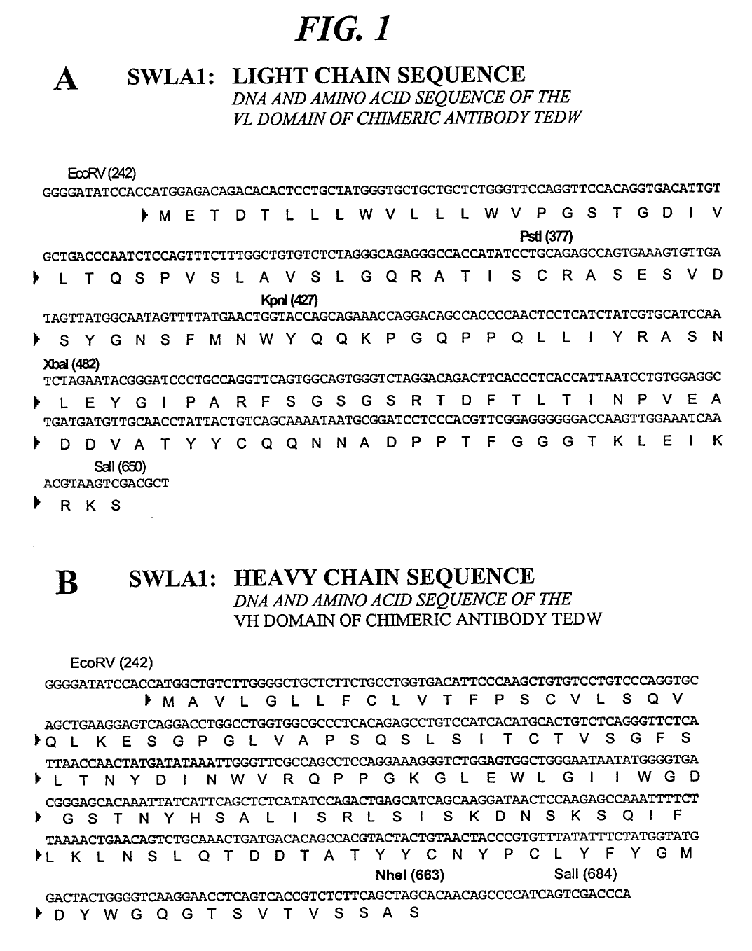

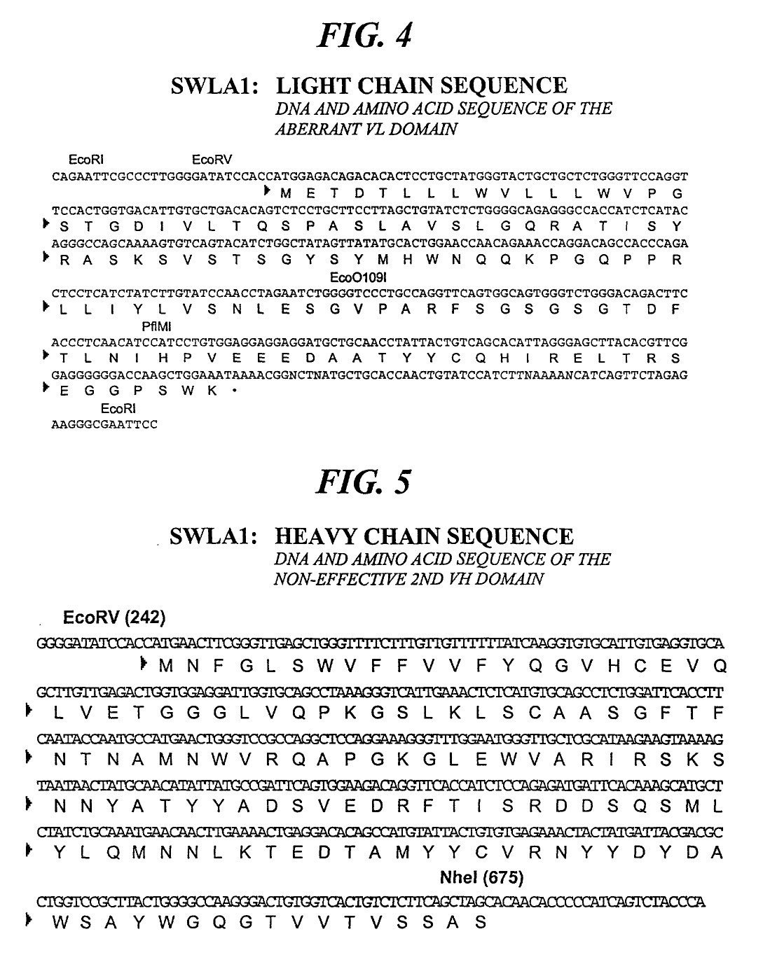

See FIG. 1 Panel A which shows the sequence coding the VL domain and the predicted amino acid sequence (SEQ ID NOS: 1 and 2) and FIG. 4 which shows the sequence coding the aberrant VL and the predicted amino acid sequence

(SEQ ID NOS: 13 and 14)
442 (SEQ ID NO: 21) 5′ GGG GAT ATC CAC ATG GAG ACA
GAC ACA CTC CTG CTA T 3′

450 (SEQ ID NO: 22) 5′ GCG TCT AGA ACT GGA TGG TGG
GAA GAT GG 3′

453 (SEQ ID NO: 23) 5′ AGC GTC GAC TTA CGT TTK ATT
TCC ARC TTK GTC CC 3′

The cloning of the heavy chain variable region (VH) resulted in finding two unique VHs both with ORFs. One VH uses signal peptide primer 440 and the other uses signal peptide primer 441 as shown below. In both reactions, the heavy chain constant region primer 451 was used. Two final PCRs were done. The first used J region primer 452 with primer 440 which generated SWLA1 VH (SEQ ID NO: 3) and the second used the same J region primer with primer 441 which produced SWLA1 2nd VH (SEQ ID NO: 15) and an aberrant non-productive VH, SWLA1 Aberrant VH (SEQ. ID NO: 17). The resulting human IgG1 expression vectors carrying the two different VHs generated are named 5937 pAH (SWLA1 VH) and 5943 pAH (SWLA1 2nd VH). Only vector 5937 pAH however was found to express an effective full length VH.

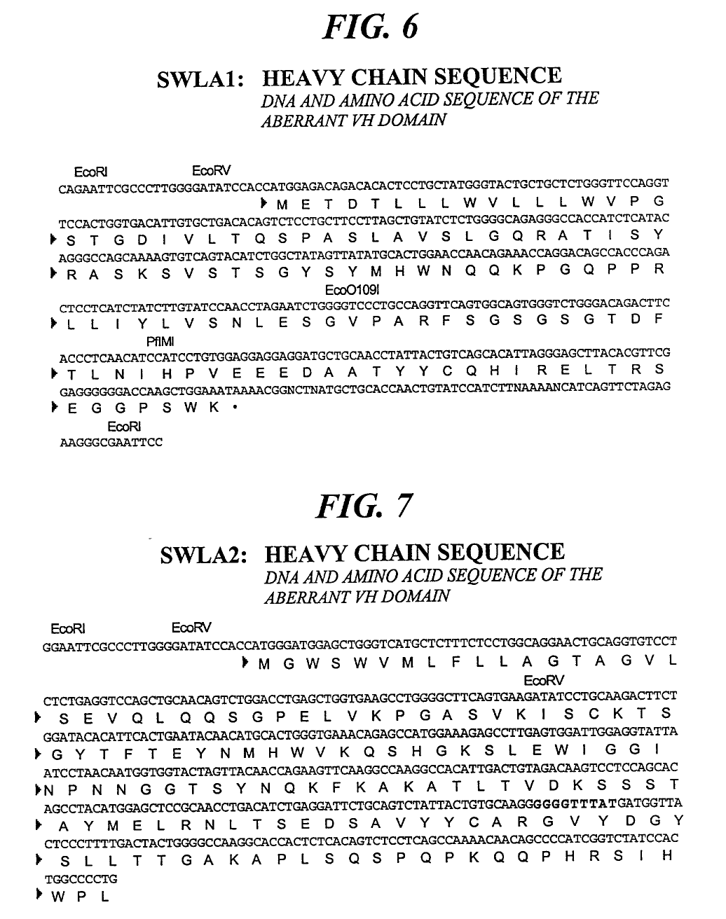

The DNA coding the VH domain and the predicted amino acid sequence are shown in FIG. 1 Panel B as SEQ ID NOS: 3 and 4. See FIG. 5 for the non-effective 2nd VH DNA and amino acid sequence (SEQ ID NOS: 15 and 16) and FIG. 6 for the DNA and amino acid sequence for the aberrant VH (SEQ ID NOS: 17 and 18).

440 (SEQ ID NO: 24) 5′ GGG GAT ATC CAC ATG RAC TTC
GGG YTG AGC TKG GTT TT 3′

441 (SEQ ID NO: 25) 5′ GGG GAT ATC CAC ATG GCT GTC
TTG GGG CTG CTC TTC T 3′

451 (SEQ ID NO: 26) 5′ AGG TCT AGA AYC TCC ACA CAC
AGG RRC CAG TGG ATA GAG 3′

452 (SEQ ID NO: 27) 5′ TGG GTC GAC WGA TGG GGS TGT
TGT GCT AGC TGA GGA GAC 3′

C. Cloning the Variable Regions from SWLA2 Cells

Two PCR products were found in cloning the VL. One product came from primers 442 and 450. The other came from primer 443 and primer 450. A unique VL with an ORF was cloned from the 443 and 450 reaction. The final PCR, which generated the SWLA2 VL (SEQ ID NO: 5), used J region primer 453 with primer 443. The resulting human kappa expression vector carrying the VL from SWLA2 is named 5938 pAG.

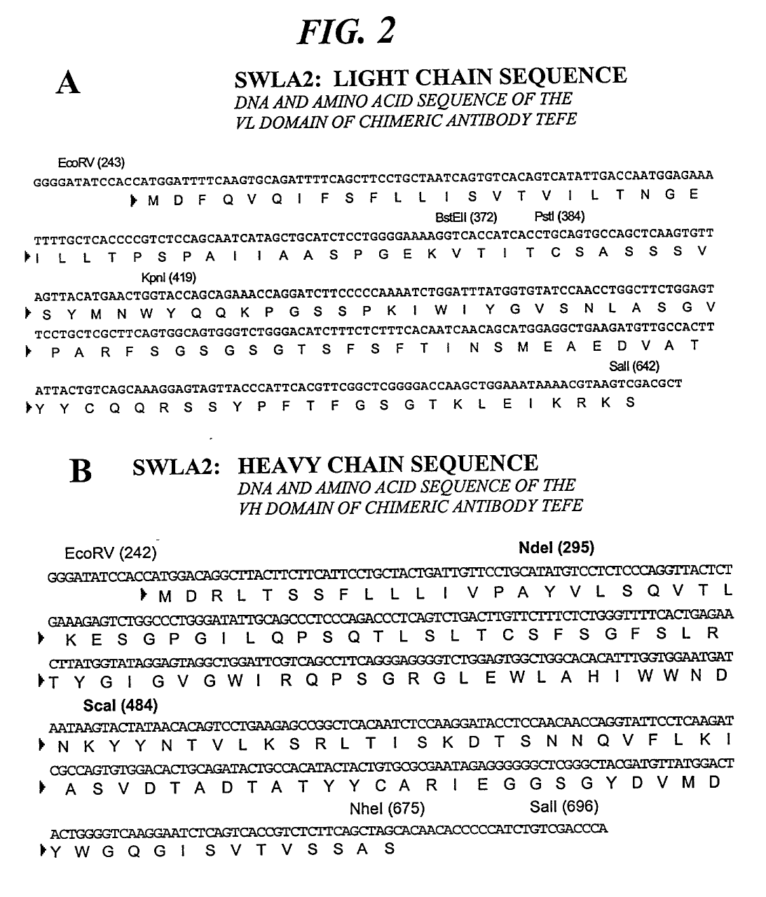

See FIG. 2 Panel A which shows the sequence coding the VL domain and the predicted amino acid sequence (SEQ II) NOS: 5 and 6).

443 (SEQ ID NO: 28) 5′ GGG GAT ATC CAC ATG GAT TTT
CAA GTG CAG ATT TTC AG 3′

Two PCR products were also found in cloning the VH. One product came from primers 439 and 451. The other product came from primers 440 and 451. The transcript from the former reaction turned out to be aberrant, SWLA2 Aberrant VH (SEQ ID NO: 19). The transcript from the latter reaction was missing part of its 5′ sequence. After aligning this sequence to several similar known VHs, a new leader signal peptide primer 843 was designed as shown below. The final PCR product SWLA2 VH (SEQ ID NO: 7) was generated with primer 843 with J region primer 452. The resulting human IgG1 expression vector carrying the VH from SWLA2 is named 5939 pAH.

The DNA coding the VH domain and the predicted amino acid sequence are shown in FIG. 2 Panel B as SEQ ID NOS: 7 and 8. See FIG. 7 for the DNA and amino acid sequence for the aberrant VH (SEQ ID NOS: 19 and 20).

439 (SEQ ID NO: 29) 5′ GGG GAT ATC CAC ATG GRA TGS
AGC TGK GTM ATS CTC TT 3′

843 (SEQ ID NO: 30) 5′ GGG ATA TCC ACC ATG GRC AGR
CTT ACW TYY TCA TTC CTG 3′

D. Cloning the Variable Regions from SWLA3 Cells

The only VL PCR product came from primer combination 442 and 450. Once again the PCR product was digested with PflMI to enrich for non-aberrant transcripts. This procedure didn't help. Another enzyme Eco0109I was used similarly and one transcript was found with the 5′ end missing. The sequence was compared to the known database and a new signal peptide primer 826 was designed as shown below. This primer 826 was then used with J region primer 835 shown below to yield the final PCR product SWLA3 VL (SEQ ID NO: 9). It was cloned into a human kappa expression vector and named 5940 pAG.

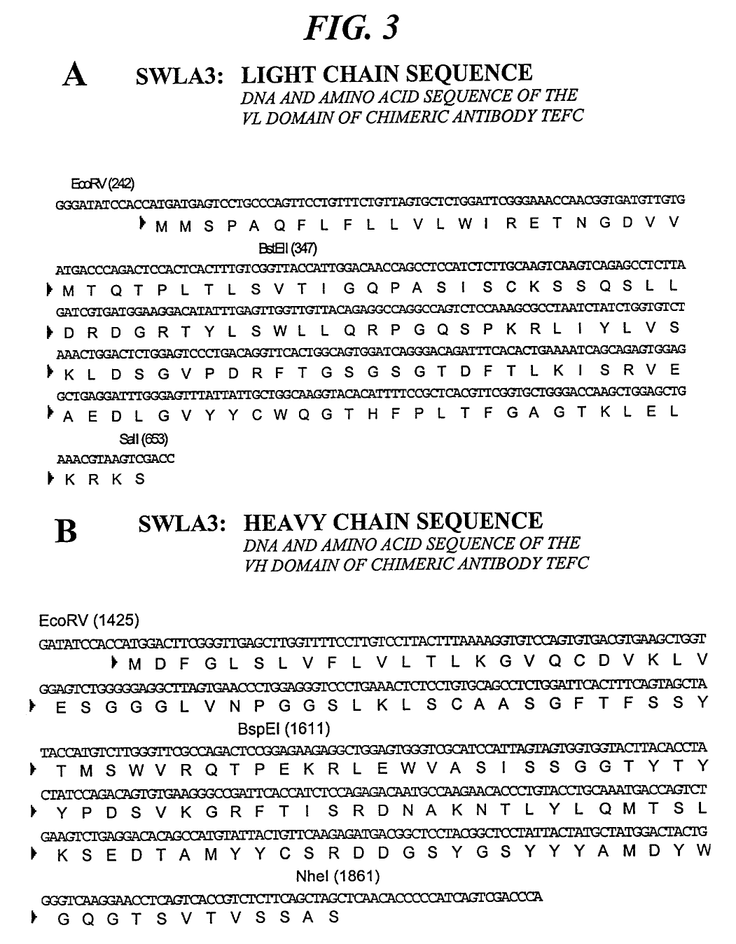

See FIG. 3 Panel A which shows the sequence coding the VL domain and the predicted amino acid sequence (SEQ ID NOS: 9 and 10).

826 (SEQ ID NO:31)5′ GGG GAT ATC CAC ATG ATG AGT CCT GCC
CAG TTC C3′

835 (SEQ ID NO:32) 5′ GGT CGA CTT AGC TTT CAG CTC CAG CTT
GGT 3′

The only VH PCR product was obtained from primer combination 440 and 451. The final PCR reaction used primer 440 and J region primer 452 to generate SWLA3 VH (SEQ ID NO: 11). The VH was cloned into a human IgG1 expression vector and named 5941 pAH.

The DNA coding the VH domain and the predicted amino acid sequence are shown in FIG. 3 Panel B as SEQ ID NOS: 11 and 12.

4. Generating Murine/Human Chimeric Genes Which Encode Humanized Monoclonal Antibodies Against S. mutans.

(i) DNA was prepared from the expression vectors and from the plasmid containing the correct V regions. See Current Protocols in Imunology, Section 2.12.1 (1994) for detailed information about the vectors that express the light and heavy chain constant regions.

(ii) The expression vector was digested with the appropriate restriction enzyme. The digests were then electrophoresed on an agarose gel to isolate the appropriate sized fragment.

(iii) The plasmid containing the cloned V region was also digested and the appropriate DNA fragment containing the V region was isolated from an agarose gel.

(iv) The V region and expression vector were then mixed together, T4 DNA ligase was added and the reaction mixture was incubated at 16° C. over night.

(v) Competent cells were transfected with the ligation mixture and the clones expressing the correct ligation sequence were selected. Restriction mapping was used to confirm the correct structure.

5. Transfecting Eukarvotic Cells

10 micrograms of DNA from each expression vector was linearized by BSPC 1 (Stratagene, PvuI isoschizomer) digestion and 1×10⁷ myeloma cells (Sp2/0 or NSO/1) were cotransfected by electroporation. Prior to transfection the cells were washed with cold PBS, then resuspended in 0.9 ml of the same cold buffer and placed in a 0.4 cm electrode gap electroporation cuvette. 960 microF and 200V were used for electroporation. The shocked cells were then incubated on ice in IMDM medium (Gibco, N.Y.) with 10% calf serum.

The transfected cells were plated into 96 well plates at a concentration of 10⁴ cells/well. Selective medium including selective drugs such as histidinol or mycophenolic acid were used to select the cells which contain expression vectors. After 12 days, the supernatants from growing clones were tested for antibody production.

6. Analyses of Recombinant Antibodies

ELISA assay was used to identify transfectomas that secrete human IgG antibodies. 100 μl of 5 μg/ml goat anti-human IgG was added to each well of a 96-well ELISA plate and incubated overnight. The plate was washed several times with PBS and blocked with 3% BSA. Supernatants from above growing clones were added to the plate for 2 hours at room temperature. Plates were then washed and anti-human kappa antibody labeled with alkaline phosphatase diluted 1:10,00 in 1% BSA was added for 1 hour at 37° C. Plates were washed with PBS and p-NPP in diethanolamine buffer (9.6% diethanolamine, 0.24 mM MgCl₂, pH 9.8) was added. Color development at OD₄₀₅ was indicative of cells producing H₂L₂.

For the supernatants that produce humanized IgG constant regions, their reactivity with S. mutans was tested as described in Shi et al., Hybridoma 17:365-371 (1998). Briefly, bacteria strains listed in Table 1 were grown in various media suggested by the American Type Culture Collection. The anaerobic bacteria were grown in an atmosphere of 80% N₂, 10% CO₂, and 10% H₂ at 37° C. The specificity of antibodies to various oral bacteria was assayed with ELISA assays. Bacteria were diluted in PBS to OD₆₀₀=0.5, and added to duplicate wells (100 μl) in 96 well PVC ELISA plates preincubated for 4 h with 100 μl of 0.02 mg/ml Poly-L-lysine. These antigen-coated plates were incubated overnight at 4° C. in a moist box then washed 3 times with PBS and blocked with 0.5% fetal calf serum in PBS and stored at 4° C. 100 μl of chimeric antibodies at 50 μg/ml were added to the appropriate wells of the antigen plates, incubated for 1 h at RT, washed 3 times with PBS-0.05% Tween 20, and bound antibody detected by the addition of polyvalent goat-anti-human IgG antibody conjugated with alkaline phosphatase diluted 1:1000 with PBS-1% fetal calf serum. After the addition of the substrate, 1 mg/ml p-nitrophenyl phosphate in carbonate buffer (15 mM Na₂CO₃, 35 mM NaH₂CO₃, 10 mM MgCl₂ pH 9.6), the color development after 15 min was measured in a EIA reader at 405 nm. “+” means OD405>1.0; “−” means OD405<0.05. The negative control is <0.05. Chimeric antibodies used are TEDW (derived from SWLA1), TEFE (derived from SWLA2) and TEFC (derived from SWLA3). The results are given in Table 1.

TABLE 1
Reactivity of Chimeric Antibodies to Various Oral Bacterial Strains

| Oral Bacteria | Strains | Chimeric antibodies |
|---|---|---|
| S. mutans | AATCC25175 | + |
| | LM7 | + |
| | OMZ175 | + |
| S. Mitis | ATCC49456 | − |
| S. rattus | ATCC19645 | − |
| S. sanguis | ATCC49295 | − |
| S sobrinus | ATCC6715-B | − |
| S. sobrinus | ATCC33478 | − |
| L. acidophilus | ATCC4356 | − |
| L. casei | ATCC11578 | − |
| L. plantarum | ATCC14917 | − |
| L. salivarius | ATCC11742 | − |
| A. actinomycetemcomitans | ATCC33384 | − |
| A. naeslundi | ATCC12104 | − |
| A. viscosus | ATCC19246 | − |
| Fusobacterium nucleatum | ATCC25586 | − |
| Porphyromonas gingivalis | ATCC33277 | − |

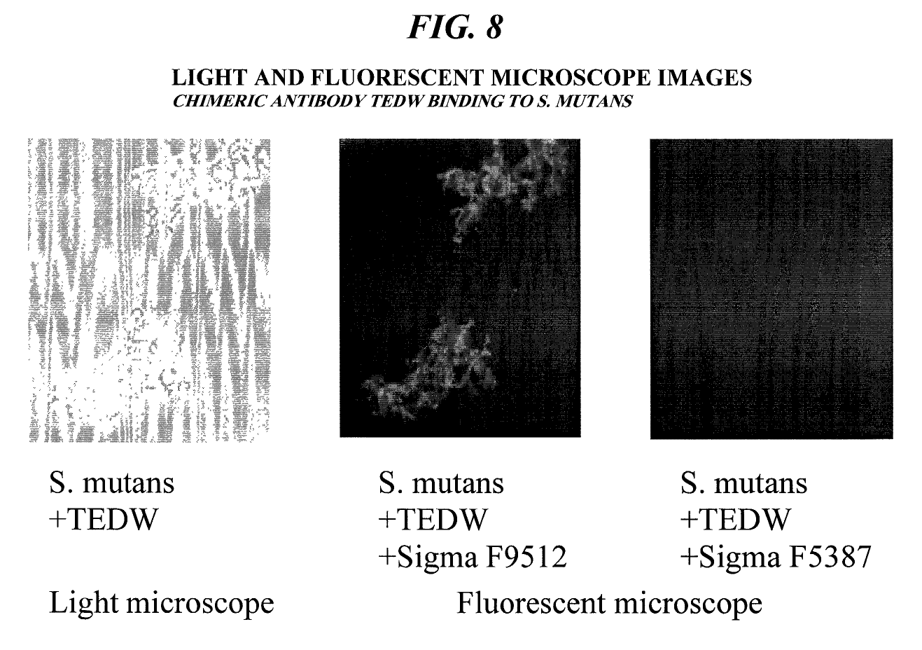

FIG. 8 shows fluorescent microscopy images generated using the chimeric TEDW antibody derived from SWLA1. S. mutans ATCC25175 was grown in Brain-Heart Infusion medium in an atmosphere of 80% N₂, 10% CO₂, and 10% H₂ at 37° C. Bacteria were then washed and resuspended in PBS buffer, mixed with various antibodies and examined with light microscopy or fluorescent microscopy. Referring to FIG. 8: Left, chimeric antibodies bind and agglutinate S. mutans cells; middle, chimeric antibodies interact with goat, FITC conjugated anti-human IgG (Fc specific) antibody (Sigma F9512) to give fluorescent image of S. mutans; right, chimeric antibodies do not react with goat, FITC conjugated anti-mouse IgG (Fc specific) antibody (Sigma F5387) and give no fluorescent image of S. mutans. Chimeric antibodies TEFE and TEFC were also used and produced results consistent with the TEDW chimeric antibody.

Results from both the flow cytometry and florescent microscopy experiments indicate that each chimeric antibody (TEDW, TEFE, and TEFC) contained both a human IgG constant region and a variable region capable of specifically recognizing S. mutans.

7. Expressing Monoclonal Antibodies to S. mutans in Transformed Organisms

A. Producing Human or Humanized Monoclonal Antibodies in Animal Cells

The heavy and light chain of a human IgG gene are separately introduced or cotransfected into an animal cell line (such as Sp2/0) using electroporation. The transfected cells are plated onto a microtiter plate and incubated at 37° C. in a 5% CO₂ atmosphere in medium containing 10% fetal bovine serum. After a 48 h incubation, the cells are grown in selection medium containing histidinol or mycophenolic acid. The supernatants of drug-resistant cells are collected and screened for immuno-reactivity against S. mutans using the ELISA or precipitation assays mentioned above.

B. Producing Human or Humanized Monoclonal Antibodies in Edible Plants

Transgenic plants have been recognized as very useful systems to produce large quantities of foreign proteins at very low cost. Expressing human or humanized monoclonal antibodies against S. mutans in edible plants (vegetables or fruits) allows direct application of plant or plant extracts to the mouth to treat existing dental caries and to prevent future bacterial infection. The choice of transgenic, edible plants includes, but is not limited to, potato, tomato, broccoli, corn, and banana.

Presented here are the procedures to produce transgenic Arabidopsis, an edible plant closely related to Brassica species including common vegetables such as cabbage, cauliflower and broccoli. It is chosen because many genetic and biochemical tools have been well developed for this plant. There are several strategies to express IgG in this plant. One strategy is to first introduce the human IgG genes encoding the heavy chain and light chain to two separate transgenic lines. The two genes are brought together by genetic crossing and selection. Other methods involve sequential transformation, in which transgenic lines transformed with one IgG gene are re-transformed with the second gene. Alternatively, genes encoding the heavy chain and light chain are cloned into two different cloning sites in the same T-DNA transformation vector under the control of two promoters, and the expression of both genes can be achieved by the transformation of a single construct to plant. Technically, the separate transformation method is the simplest one and it usually results in higher antibody yield. Therefore, we present this strategy here. It is possible to transform other plants using similar techniques.

The DNA fragments encoding the heavy and light chains of a human IgG gene are separately cloned into a Ti plasmid of Agrobacterium tumefaciens. The plasmid contains a promoter to express human heavy and light chains of IgG in Arabidopsis thaliana, an antibiotic marker for selection in Agrobacterium tumefaciens and an herbicide resistance gene for transformation selection in Arabidopsis. An Agrobacterium tumefaciens strain is transformed with these plasmids, grown to late log phase under antibiotic selection, and resuspended in infiltration medium described by Bethtold et al. (C.R. Acad. Sci. Paris Life Sci. 316:1194-1199, 1993).

Transformation of Arabidopsis by Ti-plasmid containing Agrobacterium tumefaciens is performed through vacuum infiltration. Entire plants of Arabidopsis are dipped into the bacterial suspension. The procedure is performed in a vacuum chamber. Four cycles of 5 min vacuum (about 40 cm mercury) are applied. After each application, the vacuum is released and reapplied immediately. After infiltration, plants are kept horizontally for 24 h in a growth chamber. Thereafter, the plants are grown to maturity and their seeds are harvested. The harvested seeds are germinated under unselective growth condition until the first pair of true leaves emerged. At this stage, plants are sprayed with the herbicide Basta at concentration of 150 mg/l in water. The aribidopsis plants containing transformed Ti plasmids are resistant to the herbicide while the untransformed plants are bleached and killed. Such a selection continues to the second generation of the plants. For the resistant plants, total genomic DNA is isolated and probed with the DNA fragments encoding heavy and light chains of the IgG gene. The plant extracts from the positive transformants are prepared and screened for the expression of human IgG protein with Western blot using antibodies against heavy and light chains of constant regions of human IgG.

The plants expressing human IgG heavy chain are sexually crossed with plants expressing human IgG light chain to produce progeny expressing both chains. Western blotting is used to screen the both heavy and light chains. Extracts from positive transformants are collected and screened for immuno-reactivity against S. mutans using the ELISA or precipitation assays mentioned above.

8. Using Human or Humanized Monoclonal Antibodies Against S. mutans to Treat or Prevent Human Dental Caries

With the successful completion of the above studies, humanized monoclonal antibodies against S. mutans are obtained. The plant tissue is tested for efficacy.

Plant tissue extracts containing monoclonal antibodies to S. mutans are mixed with various concentrations of S. mutans in the presence and absence of purified human complement components or purified human polymorphonuclear neutrophilic leukocytes. After a two hour incubation, the mixtures are plated onto BHI plates to examine the bactericidal activity.

Using the artificial plaque formation system developed by Wolinsky et al., J. Dent. Res. 75:816-822 (1996), plant tissue extracts containing monoclonal antibodies are used to examine the ability of the expressed monoclonal antibodies to kill S. mutans in saliva or in existing dental plaques on artificial dental enamel. Analogous techniques are used to examine the ability to prevent the formation of dental plaques.

Human clinical trials are performed using these monoclonal antibodies produced through animal cells or plants. Human volunteers are treated with or without these human monoclonal antibodies against S. mutans. Then the level of S. mutans in saliva and in dental plaques is examined. The correlation between present and future dental caries in relationship with treatment of monoclonal antibodies is also examined.

It should be understood that the foregoing examples are for illustrative purposes only, and are not intended to limit the scope of applicants' invention which is set forth in the claims appearing below.

What is claimed is:

1. A method for treatment and prevention of dental caries in a mammal comprising oral administration of a genetically engineered antibody, wherein the variable region of the antibody specifically binds to a cariogenic organism and the constant region of the antibody engages the humoral immune effector systems.

2. The method for treatment and prevention of dental caries of claim 1 wherein the cariogenic organism is Streptococcus mutans.

3. The method for treatment and prevention of dental caries of claim 2 wherein the variable region of the light chain of the antibody comprises the nucleic acid sequence of SEQ ID NO: 1.

4. The method for treatment and prevention of dental caries of claim 2 wherein the variable region of the heavy chain of the antibody comprises the nucleic acid sequence of SEQ ID NO: 3.

5. The method for treatment and prevention of dental caries of claim 2 wherein the variable region of the light chain of the antibody comprises the nucleic acid sequence of SEQ ID NO: 5.

6. The method for treatment and prevention of dental caries of claim 2 wherein the variable region of the heavy chain of the antibody comprises the nucleic acid sequence of SEQ ID NO: 7.

7. The method for treatment and prevention of dental caries of claim 2 wherein the variable region of the light chain of the antibody comprises the nucleic acid sequence of SEQ ID NO: 9.

8. The method for treatment and prevention of dental caries of claim 2 wherein the variable region of the heavy chain of the antibody comprises the nucleic acid sequence of SEQ ID NO: 11.

9. The method for treatment and prevention of dental caries wherein the variable region of the light chain of the antibody of claim 2 comprises the amino acid sequence of SEQ ID NO: 2.

10. The method for treatment and prevention of dental caries wherein the variable region of the heavy chain of the antibody of claim 2 comprises the amino acid sequence of SEQ ID NO: 4.

11. The method for treatment and prevention of dental caries wherein the variable region of the light chain of the antibody of claim 2 comprises the amino acid sequence of SEQ ID NO: 6.

12. The method for treatment and prevention of dental caries wherein the variable region of the heavy chain of the antibody of claim 2 comprises the amino acid sequence of SEQ ID NO: 8.

13. The method for treatment and prevention of dental caries wherein the variable region of the light chain of the antibody of claim 2 comprises the amino acid sequence of SEQ ID NO: 10.

14. The method for treatment and prevention of dental caries wherein the variable region of the heavy chain of the antibody of claim 2 comprises the amino acid sequence of SEQ ID NO: 12.

15. A method for treatment and prevention of dental caries in a mammal comprising oral administration of a purified antibody, wherein the variable region of the antibody specifically binds to a cariogenic organism and the constant region of the antibody engages the humoral immune effector systems.

16. The method for treatment and prevention of dental caries of claim 15 wherein the cariogenic organism is Streptococcus mutans.

17. The method for treatment and prevention of dental caries of claim 15 wherein the mammal is a human.

18. The method for treatment and prevention of dental caries of claim 17 wherein the purified antibody is produced through the steps of:
a) immunizing mice which have been genetically altered to produce human antibodies with at least one cariogenic organism;
b) generating hybridomas which secrete antibodies specific to at least one cariogenic organism; and
c) isolating the antibodies of step b).

19. The method for treatment and prevention of dental caries of claim 17 wherein the purified antibody is produced through the steps of:
a) immunizing isolated human B lymphocytes in vitro with at least one cariogenic organism;
b) generating hybridomas which secrete antibodies specific to at least one cariogenic organism;
c) isolating the antibodies of step b).

20. The method for treatment and prevention of dental caries of claim 17 wherein the purified antibody is produced through the steps of:
a) isolating B lymphocytes from humans with an acute infection of at least one cariogenic organism;
b) generating hybridomas which secrete antibodies specific to at least one cariogenic organism; and
c) isolating the antibodies of step b).

21. The method for treatment and prevention of dental caries of claim 17 wherein the purified antibody is produced through the steps of:
a) isolating the genetic sequence that codes for the expression of said variable region;
b) cloning the genetic sequence that codes for the expression of said variable region;
c) linking the genetic sequence that codes for the expression of said variable region to the genetic sequence that codes for the expression of said constant region;
d) expressing said linked sequence; and
e) isolating the expressed antibodies of step d).

22. The method for treatment and prevention of dental caries of claim 21 wherein step a) is accomplished by screening a phage display random library.

23. The method for treatment and prevention of dental caries of claim 21 wherein the genetic sequence that codes for the expression of said constant region in step c) is derived from IgG or IgM antibodies.

24. The method for treatment and prevention of dental caries of claim 21 wherein the expression of said linked sequence in step d) is conducted in an expression system selected from a group comprising animal, human, chicken egg, or plant.


SEQUENCE LISTING (32 sequences)

SEQ ID NO: 1 - DNA, 420 bp, Murine
  CDS (14)..(415)
ggggatatccaccatggagacagacacactcctgctatgggtgctgctgctctgggttccaggttccacaggtgacattgtgctgacccaatctccagtttctttggctgtgtctctagggcagagggccaccatatcctgcagagccagtgaaagtgttgatagttatggcaatagttttatgaactggtaccagcagaaaccaggacagccaccccaactcctcatctatcgtgcatccaatctagaatacgggatccctgccaggttcagtggcagtgggtctaggacagacttcaccctcaccattaatcctgtggaggctgatgatgttgcaacctattactgtcagcaaaataatgcggatcctcccacgttcggaggggggaccaagttggaaatcaaacgtaagtcgacgct

SEQ ID NO: 2 - protein, 134 aa, Murine
METDTLLLWVLLLWVPGSTGDIVLTQSPVSLAVSLGQRATISCRASESVDSYGNSFMNWYQQKPGQPPQLLIYRASNLEYGIPARFSGSGSRTDFTLTINPVEADDVATYYCQQNNADPPTFGGGTKLEIKRKS

SEQ ID NO: 3 - DNA, 454 bp, Murine
  CDS (14)..(430)
ggggatatccaccatggctgtcttggggctgctcttctgcctggtgacattcccaagctgtgtcctgtcccaggtgcagctgaaggagtcaggacctggcctggtggcgccctcacagagcctgtccatcacatgcactgtctcagggttctcattaaccaactatgatataaattgggttcgccagcctccaggaaagggtctggagtggctgggaataatatggggtgacgggagcacaaattatcattcagctctcatatccagactgagcatcagcaaggataactccaagagccaaattttcttaaaactgaacagtctgcaaactgatgacacagccacgtactactgtaactacccgtgtttatatttctatggtatggactactggggtcaaggaacctcagtcaccgtctcttcagctagcacaacagccccatcagtcgaccca

SEQ ID NO: 4 - protein, 139 aa, Murine
MAVLGLLFCLVTFPSCVLSQVQLKESGPGLVAPSQSLSITCTVSGFSLTNYDINWVRQPPGKGLEWLGIIWGDGSTNYHSALISRLSISKDNSKSQIFLKLNSLQTDDTATYYCNYPCLYFYGMDYWGQGTSVTVSSAS

SEQ ID NO: 5 - DNA, 411 bp, Murine
  CDS (14)..(406)
ggggatatccaccatggattttcaagtgcagattttcagcttcctgctaatcagtgtcacagtcatattgaccaatggagaaattttgctcaccccgtctccagcaatcatagctgcatctcctggggaaaaggtcaccatcacctgcagtgccagctcaagtgttagttacatgaactggtaccagcagaaaccaggatcttcccccaaaatctggatttatggtgtatccaacctggcttctggagttcctgctcgcttcagtggcagtgggtctgggacatctttctctttcacaatcaacagcatggaggctgaagatgttgccacttattactgtcagcaaaggagtagttacccattcacgttcggctcggggaccaagctggaaataaaacgtaagtcgacgct

SEQ ID NO: 6 - protein, 131 aa, Murine
MDFQVQIFSFLLISVTVILTNGEILLTPSPAIIAASPGEKVTITCSASSSVSYMNWYQQKPGSSPKIWIYGVSNLASGVPARFSGSGSGTSFSFTINSMEAEDVATYYCQQRSSYPFTFGSGTKLEIKRKS

SEQ ID NO: 7 - DNA, 465 bp, Murine
  CDS (13)..(441)
gggatatccaccatggacaggcttacttcttcattcctgctactgattgttcctgcatatgtcctctcccaggttactctgaaagagtctggccctgggatattgcagccctcccagaccctcagtctgacttgttctttctctgggttttcactgagaacttatggtataggagtaggctggattcgtcagccttcagggaggggtctggagtggctggcacacatttggtggaatgataataagtactataacacagtcctgaagagccggctcacaatctccaaggatacctccaacaaccaggtattcctcaagatcgccagtgtggacactgcagatactgccacatactactgtgcgcgaatagaggggggctcgggctacgatgttatggactactggggtcaaggaatctcagtcaccgtctcttcagctagcacaacacccccatctgtcgaccca

SEQ ID NO: 8 - protein, 143 aa, Murine
MDRLTSSFLLLIVPAYVLSQVTLKESGPGILQPSQTLSLTCSFSGFSLRTYGIGVGWIRQPSGRGLEWLAHIWWNDNKYYNTVLKSRLTISKDTSNNQVFLKIASVDTADTATYYCARIEGGSGYDVMDYWGQGISVTVSSAS

SEQ ID NO: 9 - DNA, 420 bp, Murine
  CDS (13)..(417)
gggatatccaccatgatgagtcctgcccagttcctgtttctgttagtgctctggattcgggaaaccaacggtgatgttgtgatgacccagactccactcactttgtcggttaccattggacaaccagcctccatctcttgcaagtcaagtcagagcctcttagatcgtgatggaaggacatatttgagttggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgctcacgttcggtgctgggaccaagctggagctgaaacgtaagtcgacc

SEQ ID NO: 10 - protein, 135 aa, Murine
MMSPAQFLFLLVLWIRETNGDVVMTQTPLTLSVTIGQPASISCKSSQSLLDRDGRTYLSWLLQRPGQSPKRLIYLVSKLDSGVPDRFTGSGSGTDFTLKISRVEAEDLGVYYCWQGTHFPLTFGAGTKLELKRKS

SEQ ID NO: 11 - DNA, 466 bp, Murine
  CDS (11)..(442)
gatatccaccatggacttcgggttgagcttggttttccttgtccttactttaaaaggtgtccagtgtgacgtgaagctggtggagtctgggggaggcttagtgaaccctggagggtccctgaaactctcctgtgcagcctctggattcactttcagtagctataccatgtcttgggttcgccagactccggagaagaggctggagtgggtcgcatccattagtagtggtggtacttacacctactatccagacagtgtgaagggccgattcaccatctccagagacaatgccaagaacaccctgtacctgcaaatgaccagtctgaagtctgaggacacagccatgtattactgttcaagagatgacggctcctacggctcctattactatgctatggactactggggtcaaggaacctcagtcaccgtctcttcagctagctcaacacccccatcagtcgaccca

SEQ ID NO: 12 - protein, 144 aa, Murine
MDFGLSLVFLVLTLKGVQCDVKLVESGGGLVNPGGSLKLSCAASGFTFSSYTMSWVRQTPEKRLEWVASISSGGTYTYYPDSVKGRFTISRDNAKNTLYLQMTSLKSEDTAMYYCSRDDGSYGSYYYAMDYWGQGTSVTVSSAS

SEQ ID NO: 13 - DNA, 481 bp, Murine
  CDS (28)..(411)
  variation (420)..(420): atgc
  variation (449)..(449): atgc
  variation (454)..(454): atgc
  variation (423)..(423): atgc
cagaattcgcccttggggatatccaccatggagacagacacactcctgctatgggtactgctgctctgggttccaggttccactggtgacattgtgctgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaataaaacggnctnatgctgcaccaactgtatccatcttnaaaancatcagttctagagaagggcgaattcc

SEQ ID NO: 14 - protein, 128 aa, Murine
METDTLLLWVLLLWVPGSTGDIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWK

SEQ ID NO: 15 - DNA, 466 bp, Murine
  CDS (14)..(442)
ggggatatccaccatgaacttcgggttgagctgggttttctttgttgttttttatcaaggtgtgcattgtgaggtgcagcttgttgagactggtggaggattggtgcagcctaaagggtcattgaaactctcatgtgcagcctctggattcaccttcaataccaatgccatgaactgggtccgccaggctccaggaaagggtttggaatgggttgctcgcataagaagtaaaagtaataactatgcaacatattatgccgattcagtggaagacaggttcaccatctccagagatgattcacaaagcatgctctatctgcaaatgaacaacttgaaaactgaggacacagccatgtattactgtgtgagaaactactatgattacgacgcctggtccgcttactggggccaagggactgtggtcactgtctcttcagctagcacaacacccccatcagtctaccca

SEQ ID NO: 16 - protein, 143 aa, Murine
MNFGLSWVFFVVFYQGVHCEVQLVETGGGLVQPKGSLKLSCAASGFTFNTNAMNWVRQAPGKGLEWVARIRSKSNNYATYYADSVEDRFTISRDDSQSMLYLQMNNLKTEDTAMYYCVRNYYDYDAWSAYWGQGTVVTVSSAS

SEQ ID NO: 17 - DNA, 518 bp, Murine
  CDS (29)..(376)
  variation (459)..(459): atgc
  variation (482)..(482): atgc
  variation (490)..(491): atgc
gcagaattcgcccttggggatatccaccatggctgtcttggggctgctcttctgcctggtgacattcccaagctgtgtcctgtcccaggtgcagctgaaggactcaggacctggcctggtggcgccctcacagagcctgtccatcacatgcactgtctcagggttctcattaaccaactatgatataaattgggttcgccagcctccaggaaagggtctggagtggctgggaataatatggggtgacgggagcacaaattatcattcagctctcatatccagactgagcatcagcaaggataactccaagagccaaattttcttaaaactgaacagtctgcaaactgatgacacagccacgtactactgtttattgtaactacccgtgtttatatttctatggtatggactactggggtcaaggaacctcagtcaccgtctcctcagccaaaacaacanccccatcggtctatccactggcncctgtganngagattctaaacctaagggcgaattcc

SEQ ID NO: 18 - protein, 116 aa, Murine
MAVLGLLFCLVTFPSCVLSQVQLKDSGPGLVAPSQSLSITCTVSGFSLTNYDINWVRQPPGKGLEWLGIIWGDGSTNYHSALISRLSISKDNSKSQIFLKLNSLQTDDTATYYCLL

SEQ ID NO: 19 - DNA, 482 bp, Murine
  CDS (27)..(482)
ggaattcgcccttggggatatccaccatgggatggagctgggtcatgctctttctcctggcaggaactgcaggtgtcctctctgaggtccagctgcaacagtctggacctgagctggtgaagcctggggcttcagtgaagatatcctgcaagacttctggatacacattcactgaatacaacatgcactgggtgaaacagagccatggaaagagccttgagtggattggaggtattaatcctaacaatggtggtactagttacaaccagaagttcaaggccaaggccacattgactgtagacaagtcctccagcacagcctacatggagctccgcaacctgacatctgaggattctgcagtctattactgtgcaaggggggtttatgatggttactcccttttgactactggggccaaggcaccactctcacagtctcctcagccaaaacaacagccccatcggtctatccactggcccctg

SEQ ID NO: 20 - protein, 152 aa, Murine
MGWSWVMLFLLAGTAGVLSEVQLQQSGPELVKPGASVKISCKTSGYTFTEYNMHWVKQSHGKSLEWIGGINPNNGGTSYNQKFKAKATLTVDKSSSTAYMELRNLTSEDSAVYYCARGVYDGYSLLTTGAKAPLSQSPQPKQQPHRSIHWPL

SEQ ID NO: 21 - DNA, 37 bp, synthetic construct
ggggatatccacatggagacagacacactcctgctat

SEQ ID NO: 22 - DNA, 29 bp, synthetic construct
gcgtctagaactggatggtgggaagatgg

SEQ ID NO: 23 - DNA, 35 bp, synthetic construct
agcgtcgacttacgtttkatttccarcttkgtccc

SEQ ID NO: 24 - DNA, 38 bp, synthetic construct
ggggatatccacatgracttcgggytgagctkggtttt

SEQ ID NO: 25 - DNA, 37 bp, synthetic construct
ggggatatccacatggctgtcttggggctgctcttct

SEQ ID NO: 26 - DNA, 39 bp, synthetic construct
aggtctagaayctccacacacaggrrccagtggatagac

SEQ ID NO: 27 - DNA, 39 bp, synthetic construct
tgggtcgacwgatggggstgttgtgctagctgaggagac

SEQ ID NO: 28 - DNA, 38 bp, synthetic construct
ggggatatccacatggattttcaagtgcagattttcag

SEQ ID NO: 29 - DNA, 38 bp, synthetic construct
ggggatatccacatggratgsagctgkgtmatsctctt

SEQ ID NO: 30 - DNA, 39 bp, synthetic construct
gggatatccaccatggrcagrcttacwtyytcattcctg

SEQ ID NO: 31 - DNA, 34 bp, synthetic construct
ggggatatccacatgatgagtcctgcccagttcc

SEQ ID NO: 32 - DNA, 30 bp, synthetic construct
ggtcgacttacgtttcagctccagcttggt